(19) United States
(12) Patent Application Publication
(10) Pub. No.: US 2023/0134747 A1
(43) Pub. Date: May 4, 2023

(54) COMPOSITIONS FOR THE TREATMENT OF AUTISM SPECTRUM DISORDER

(71) Applicant: STALICLA SA, Genève (CH)

(72) Inventor: Lynn DURHAM, Genève (CH)

(73) Assignee: STALICLA SA, Genève (CH)

(21) Appl. No.: 17/912,108

(22) PCT Filed: Mar. 19, 2021
(86) PCT No.: PCT/EP2021/057162

§ 371 Date: Sep. 16, 2022

(30) Foreign Application Priority Data
Mar. 19, 2020 (EP) 20164331.9

(51) Int. Cl. A61K 31/196; A61P 25/00; C12Q 1/6883

(52) U.S. Cl. CPC A61K 31/196; A61P 25/00; C12Q 1/6883; C12Q 2600/106; C12Q 2600/158

(57) ABSTRACT

The present invention relates to pharmaceutical compositions for the treatment of a subtype of autism spectrum disorder (ASD) patients. Likewise, the present invention relates to methods of diagnosing a subtype of ASD and methods for identifying patients responding a specific treatment of ASD.

### FIELD OF THE INVENTION

The present invention relates to pharmaceutical compositions for the treatment of a subtype of autism spectrum disorder (ASD) patients. Likewise, the present invention relates to methods of diagnosing a subtype of ASD and methods for identifying patients responding a specific treatment of ASD.

### BACKGROUND OF THE INVENTION

Autism spectrum disorder (ASD) is one of the most prevalent and disabling neurodevelopmental disorders (NDD). The prevalence of ASD is currently estimated at 1% in the world population and 1 in 59 school-aged children in the US (1 in 37 boys and 1 in 151 girls) (Baio et al. Prevalence of Autism Spectrum Disorder Among Children Aged 8 Years—Autism and Developmental Disabilities Monitoring Network, 11 Sites, United States, 2014. MMWR Surveill Summ. 2018 Apr. 27; 67(6):1-23). ASD is still considered a single diagnostic entity whose diagnosis remains based on observation of atypical behaviors characterized by deficits in 1) social interactions and communication, including deficits in social-emotional reciprocity; nonverbal communicative behaviors used for social interaction, and in developing, maintaining, and understanding relationships; 2) at least 4 subdomains of restricted or repetitive behaviors, including stereotyped or repetitive motor movements, insistence on sameness or inflexible adherence to routines, highly restricted and fixated interests, hyper- or hyporeactivity to sensory input, or unusual interest in the sensory aspects of the environment (Baird G, et al. Neurodevelopmental disorders. American Psychiatric Association. Diagnostic and Statistical Manual of Mental Disorders-Fifth Edition (DSM-5). Washington, D.C.: American Psychiatric Publishing, 2013: p. 31-86).

Autism is known as a “spectrum disorder” because of the wide heterogeneity in etiology, phenotype, severity and outcome. These factors contribute to a clinical heterogeneity that manifest as diverse deficits or impairments in behavioral features and communicative functioning. This marked heterogeneity of the condition has led to suggestions that rather than a single disorder, ASD, or “the autisms”, encompass multiple etiologies and distinct clinical entities. Therefore, the current behavioral-based approaches to diagnose ASD patients do not provide an efficient and precise classification in terms of genetic alterations and disrupted molecular pathways.

There is currently no approved treatment to address the core symptoms of ASD.

Sulforaphane, a potent modulator of heat shock transcription factor 1 and heat-shock proteins, has been suggested as a treatment for ASD (WO 2013/067040). However, results from in-vivo studies are not sufficient to demonstrate that it might actually benefit to the ASD patient population in clinical setting.

In a clinical setting, sulforaphane has been shown to improve behaviour in some ASD patients (Singh et al. Sulforaphane treatment of autism spectrum disorder (ASD). PNAS 2014; 111:43, 15550-5). However, the authors reported heterogeneous responses, with either significant improvement, or no changes or even worsening of behavioural symptoms in participants. Furthermore, the authors acknowledged limitations of their study. More specifically, as reported in the discussion, the authors stated that “participants with ASD whose behaviour improved during fever would also respond to sulforaphane could not be confirmed because of the unusually high prevalence of fever responders (80%) in our cohort compared with most ASD populations (35%)”. Therefore, despite interesting findings, the lack of characterization of potential responders hinders the application of sulforaphane as validated treatment.

This heterogeneity of ASD patients with regards to the treatment response (also illustrated by the arbaclofen or bumetanide trials (Veenstra-VanderWeele et al. Arbaclofen in Children and Adolescents with Autism Spectrum Disorder: A Randomized, Controlled, Phase 2 Trial. Neuropsychopharmacology. 2017 June; 42(7):1390-1398; Lemonier et al. A randomised controlled trial of bumetanide in the treatment of autism in children. Transl Psychiatry. 2012; 11; 2: e202; Lemonier et al. Effects of bumetanide on neurobehavioral function in children and adolescents with autism spectrum disorders. 2017; Translational Psychiatry volume 7, e1056)), further supports the urgent need to shift from the “one-size-fits-all” approach towards understanding molecular and genetic heterogeneity in order to provide the right treatment to the right patients.

As the underlying causes of ASD remain elusive, attempts have already been made to stratify ASD patients into smaller, more homogeneous subgroups, by focusing on specific genetic alteration signatures (Bernier et al., Disruptive CHD8 mutations define a subtype of autism early in development, Cell 2014 Jul. 17; 158 (2): 263-276; Kuechler et al. Bainbridge-Ropers syndrome caused by loss-of-function variants in ASXL3: a recognizable condition. Eur J Hum Genet. 2017 February; 25(2):183-191). Importantly, these analyses provide evidence for connections between specific genetic alterations in carriers of loss-of-function mutations in the CHD8 and ASXL3 genes and ASD. But beyond characterizing the connection between single genetic insults and observable phenotype outcomes, in order to develop drug treatments that are efficient for a specific and well-defined subset of ASD patients, it is key to also understand the underlying disrupted pathways in these patients.

In this context, a method capable of identifying subsets of idiopathic ASD patients sharing a common dysregulation pattern has been previously reported (EP19383010 and EP20164353). This approach allows to cluster patients based on common clinical signs and symptoms, which are caused by the common dysregulation pattern, and then identify treatments that are suitable for resolving the dysregulation on a genetic or molecular level.

However, given the heterogeneity of ASD, there is a need to identify further subtypes of ASD that will respond to specific treatments.

### Objective Problem to be Solved

The problem to be solved is thus the provision of means to efficiently identify a further subgroup of ASD patients and the provision of an effective treatment for this subgroup.

### SUMMARY OF INVENTION

The invention is directed to a pharmaceutical composition comprising a substance capable of inducing repolarization of the membrane of excitable cells after depolarization for use in the treatment of autism spectrum disorder (ASD) phenotype 3.

Furthermore, the invention is directed to a pharmaceutical composition comprising a substance capable of inducing repolarization of the membrane of excitable cells after depolarization for use in the treatment of ASD in a patient, wherein the patient shows at least two of the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia.

The invention is also directed to a method for dividing an ASD patient cohort into patients responding to treatment with a substance capable of inducing repolarization of the membrane of excitable cells after depolarization and patients not responding to said treatment, comprising the steps of:
providing a patient database including data sets relating to the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia; and
classifying a patient as a patient responding to treatment with a substance capable of inducing repolarization of the membrane of excitable cells after depolarization if he shows at least two of the clinical signs and symptoms of said first group.

The invention is further directed to a method for diagnosing an ASD patient with ASD phenotype 3, comprising the steps of:
providing a patient-specific data set relating to the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia; and
diagnosing the patient with ASD phenotype 3 if he shows at least two of the clinical signs and symptoms of said first group.

### DETAILED DESCRIPTION

The present invention relates to a pharmaceutical composition comprising a substance capable of inducing repolarization of the membrane of excitable cells after depolarization for use in the treatment of a specific subgroup of ASD patients, called ASD phenotype 3.

As used herein, the term autism spectrum disorder (ASD) is understood to cover a family of neurodevelopmental disorders characterized by deficits in social communication and interaction and restricted, repetitive patterns of behavior, interests or activities. In the following, the terms “autism spectrum disorder”, “autism” and “ASD” are used interchangeably.

Herein, the terms “ASD Phenotype 3” and “Phenotype 3” are used interchangeably.

The term “patient” refers to “ASD patient” and is intended to cover not only humans diagnosed as having ASD, but also humans suspected of having ASD.

The person skilled in the art is well aware of how a patient may be diagnosed with ASD.

For example, the skilled person may follow the criteria set up in “American Psychiatric Association; Diagnostic and Statistical Manual of Mental Disorders (DSM-5) Fifth edition” to give a subject a diagnosis of ASD. Likewise, ASD patients may have been diagnosed according to standardized assessments tools including but not limited to CIM-10, ICD-10, DISCO, ADI-R, ADOS or CHAT.

In other cases, patients may have a well-established DSM-IV diagnosis of autistic disorder, Asperger's disorder, or pervasive developmental disorder not otherwise specified (PDD-NOS).

Additionally, the present invention may be useful for subjects fulfilling one or more of the following criteria: persistent deficits in social communication and social interaction across multiple contexts as manifested by the following, currently or by history; restricted, repetitive patterns of behavior, interests, or activities, as manifested by at least two of the following, currently or by history; symptoms present in the early development period (but may not become fully manifest until social demands exceed limited capacities, or maybe masked by learned strategies in later life); symptoms cause clinically significant impairment in social, occupational, or other important areas of current functioning; these disturbances are not better explained by intellectual disability (intellectual development disorder) or global development delay.

ASD may occur with or without accompanying intellectual and/or language impairment. It may be associated with a known medical or genetic condition or an environmental factor or other neurodevelopmental, mental or behavioral disorders.

ASD may occur in different severity levels which may be classified according to impairment in social communication and in terms of restricted, repetitive behavior. Importantly, the term ASD phenotype 1 is not associated with a particular severity level of ASD. The present invention may be applied to patients suffering from any severity level of ASD.

The present inventors have surprisingly identified a subgroup of ASD patients called ASD phenotype 3 which are characterized by a prolonged repolarization of the membrane of various cell types after depolarization (i.e., abnormally long lasting potentials) due to an altered expression of membrane channels or key regulators of membrane channels. So called channelopathy ultimately leads to altered release of neurotransmitters and hormones that trigger repeated membrane depolarization (Schmunk et al. Channelopathy pathogenesis in autism spectrum disorders. Front Genet. 2013 Nov. 5; 4:222). Such alterations are not restricted to neuronal cells, but also occur in other excitable cells such as beta pancreatic cells.

In ASD phenotype 3 patients, prolonged membrane depolarization causes abnormal brain development with pathological alterations in neuronal differentiation and maturation (Sohya et al. Chronic membrane depolarization-induced morphological alteration of developing neurons. Neuroscience. 2007 Mar. 2; 145(1):232-40), including a reduced number of cells expressing GABA and serotonin neurotransmitters (He at al. Prolonged membrane depolarization enhances midbrain dopamine neuron differentiation via epigenetic histone modifications. Stem Cells. 2011 November; 29(11):1861-73). This therefore leads to excessive glutamate release, which promotes inflammation (Wen et al. Excitatory amino acid glutamate: role in peripheral nociceptive transduction and inflammation in experimental and clinical osteoarthritis. Osteoarthritis Cartilage. 2015 November; 23(11):2009-16; Osikowicz et al. The glutamatergic system as a target for neuropathic pain relief. Exp Physiol. 2013 February; 98(2):372-84; Swaim et al. Platelets contribute to allograft rejection through glutamate receptor signaling. J Immunol. 2010 Dec. 1; 185(11):6999-7006) and triggers repeated membrane depolarization. In ASD phenotype 3 patients, prolonged membrane depolarization may also cause hyperinsulinemia (Daraio et al. SNAP-25b-deficiency increases insulin secretion and changes spatiotemporal profile of Ca2+oscillations in β cell networks. Sci Rep. 2017 Aug. 10; 7(1):7744). Chronic (or congenital) hyperinsulinemia leads to recurrent episodes of hypoglycemia, which decreases the capacity of energy production in the brain; this can in turn lead to failure of the ATP-dependent sodium-potassium pumps, and therefore exacerbate the excess of membrane depolarization by making the cells incapable of maintaining a stable resting membrane potential (Abend et al. Chapter 12—Neonatal Seizures. Volpe's Neurology of the Newborn (Sixth Edition) 2018, Pages 275-321.e14), thus increasing the risk for seizures (Sousa-Santos et al. Congenital hyperinsulinism in two siblings with ABCC8 mutation: same genotype, different phenotypes. Arch Endocrinol Metab. 2018 October; 62(5):560-565; Aka et al. Seizures and diagnostic difficulties in hyperinsulinism-hyperammonemia syndrome. Turk J Pediatr. 2016; 58(5):541-544). In the long term, hyperinsulinemia can lead to insulin resistance and type 2 diabetes, resulting in recurrent episodes of hyperglycemia (Shannik et al. Insulin resistance and hyperinsulinemia: is hyperinsulinemia the cart or the horse? Diabetes Care. 2008 February; 31 Suppl 2:S262-8).

It is therefore an aim of the invention to provide these patients with a novel treatment directed to mitigating the effects of prolonged membrane depolarization. This is achieved by administration of a substance capable of inducing repolarization of the cell membrane after depolarization.

Herein the term “substance capable of inducing repolarization of the cell membrane after depolarization” is understood to relate to substances that help restore the cell resting membrane potential to its physiological range by counteracting abnormally prolonged repolarizations (i.e., abnormally long lasting action potentials). Prolonged repolarization is often caused by a channelopathy and leads to an abnormal release of neurotransmitters and hormones and an excess of membrane depolarization. The person skilled in the art is well aware of how the altered ion homeostasis and membrane channel expression can modify cell membrane potential and cause dysfunction of various cellular activities such as gene expression, mitochondrial function, protein synthesis, protein trafficking, or neurotransmitter release.

In one aspect of the invention, the excitable cells are neuronal and beta pancreatic cells, i.e., the pharmaceutical composition comprises a substance capable of inducing repolarization of the membrane of neuronal and beta pancreatic cells.

Since the mechanisms described above apply in particular to the subgroup of ASD phenotype 3 patients, the invention is directed to pharmaceutical compositions comprising substances capable of inducing repolarization of the membrane of excitable cells for use in ASD phenotype 3 patients.

According to the invention, ASD phenotype 3 patients can be identified by checking for the presence of one or more clinical signs and symptoms of a first group comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia.

Herein, it is defined that a patient shows the clinical sign and symptom “gastric ulcer” if the patient has an open sore on an area of the gastric mucous membrane.

Herein, it is defined that a patient shows the clinical sign and symptom “hyperkinesia” if the patient has motor hyperactivity with excessive movement of muscles of the body as a whole.

Herein, it is defined that a patient shows the clinical sign and symptom “hyperventilation” if the patient has an increased pulmonary ventilation rate that is faster than necessary for the exchange of gases.

Herein, it is defined that a patient shows the clinical sign and symptom “tachycardia” if the patient's heart rate exceeds the normal resting state according to age as defined in Table 2.

TABLE 2
Normal pulse rates at rest, in beats per minute (BPM; U.S. Department
of Health and Human Services - National Ites of Health)

| | | | | children over | |
|---|---|---|---|---|---|
| | | | | 10 years | |
| newborn | infants | infants | children | & adults, | |
| (0-3 | (3-6 | (6-12 | (1-10 | including | well-trained |
| months old) | months) | months) | years) | seniors | adult athletes |
| 99-149 | 89-119 | 79-119 | 69-129 | 59-99 | 39-59 |

Herein, it is defined that a patient shows the clinical sign and symptom “short neck” if the patient's neck measures less than the width of 4 extended fingers of his/her palm (without considering the thumb).

Herein, it is defined that a patient shows the clinical sign and symptom “hypophosphatemia” if the patient has a serum phosphate level of less than 2.5 mg/dL (adult) or less than 4 mg/dL (children; Sharma et al. Hypophosphatemia. St Treasure Island (FL): StatPearls Publishing).

Herein, it is defined that a patient shows the clinical sign and symptom “McCune-Albright syndrome” if the patient has ever received such medical diagnosis or shows symptoms fitting the diagnosis of “McCune-Albright syndrome”.

Herein, it is defined that a patient shows the clinical sign and symptom “seizures” if the patient has ever experienced sudden attacks of excessive or synchronous neuronal activity in the brain with physical manifestations such as convulsions, sensory disturbances, or loss of consciousness.

Herein, it is defined that a patient shows the clinical sign and symptom “family history of hypertension” if the patient or a first- or second-degree family member of the patient has a history of having a systolic blood pressure (SBP) of 140 mm Hg or more, or a diastolic blood pressure (DBP) of 90 mm Hg or more (for adults; Chobanian et al. Seventh report of the Joint National Committee on Prevention, Detection, Evaluation, and Treatment of High Blood Pressure. Hypertension. 2003 December 42(6):1206-52), or if the patient or a first- or second-degree family member of the patient has a history of having blood pressure being above the 95th percentile according to age (for children; Flynn et al. Clinical Practice Guideline for Screening and Management of High Blood Pressure in Children and Adolescents. Pediatrics September 2017, 140 (3) e20171904).

Herein, it is defined that a patient shows the clinical sign and symptom “hyperkalemia” if the patient has a serum potassium level above 5.0 mEq/L (for children over the age of 2 years to adults; Simon et al. Hyperkalemia. St Treasure Island (FL): StatPearls Publishing).

Herein, it is defined that a patient shows the clinical sign and symptom “gastroesophageal reflux” if the patient suffers from a burning feeling in his/her chest or throat due to acidic stomach juices leaking backwards from the stomach into the esophagus through the lower esophageal sphincter.

Herein, it is defined that a patient shows the clinical sign and symptom “precocious puberty” if the onset of puberty happens before the age of 8 years in girls or 9 years in boys.

Herein, it is defined that a patient shows the clinical sign and symptom “hyperinsulinemia” if the patient has an elevated concentration of insulin in the blood compared to the normal/expected range as defined in Table 1.

TABLE 1
Reference ranges of insulin and glucose levels for children over
the age of 2 years to adult (Pagana et al. Mosby's Diagnostic
& Laboratory Test Reference. 14th ed. St. Louis, Mo: Elsevier; 2019;
Yorifuji. Congenital hyperinsulinism: current status and future
perspectives. Ann Pediatr Endocrinol Metab. 2014 June; 19(2): 57-68.)

| | Insulin level | | Glucose level |
|---|---|---|---|
| Hypoglycemia | <20.84 | pmol/L | <2.5 mmol/L |
| Fasting (ie., no caloric intake | <174 | pmol/L | <6.1 mmol/L |
| for at least 8 hours) | | | |
| 2 hours after glucose | 111-1153 | pmol/L | <7.8 mmol/L |
| administration | | | |

Herein, it is defined that a patient shows the clinical sign and symptom “increased inflammatory response” if the patient shows an overproduction of immune cells or proteins including Th1 cytokines, a family history of autoimmune disorders, or worsening of ASD symptoms under sickness.

Herein, it is defined that a patient shows the clinical sign and symptom “family history of cholangiocarcinoma” if the patient or a first- or second-degree family member of the patient has ever received a diagnosis “cholangiocarcinoma” or shows symptoms fitting the diagnosis of “cholangiocarcinoma”.

Herein, it is defined that a patient shows the clinical sign and symptom “hearing impairment” if he/she shows a decreased magnitude of the sensory perception of sound.

Herein, it is defined that a patient shows the clinical sign and symptom “hyperchloraemia” if the patient has a serum chloride concentration above 106 mEq/L (Morrison. Clinical Methods: The History, Physical, and Laboratory Examinations. 3rd edition. Chapter 197. Boston: Butterworths).

Herein, it is defined that a patient shows the clinical sign and symptom “family history of diabetes” if the patient or a first- or second-degree family member of the patient has ever received the diagnosis “type I or type II diabetes” or shows symptoms fitting the diagnosis of “type I or type II diabetes”.

Herein, it is defined that a patient shows the clinical sign and symptom “joint hypermobility” if patient has hypermobile joints that can move beyond the normal range of motion.

Herein, it is defined that a patient shows the clinical sign and symptom “constipation” if patient has abnormally delayed or infrequent passage of usually dry hardened feces.

Herein, it is defined that a patient shows the clinical sign and symptom “family history of breast cancer” if patient or a first- or second-degree family member of the patient has ever received a diagnosis of breast cancer or shows symptoms fitting the diagnosis of “breast cancer”.

Here it is defined that a patient shows the clinical sign and symptom “worsening of ASD core symptoms under benzodiazepines” if patient's symptoms show aggravation of ASD core symptoms such as excitement, agitation/hyperactivity, excessive movement, increased display of emotional volatility, irritability, aggression, hostility or rage and self-injurious behavior, increased talkativeness or inappropriate vocalization, screaming and/or crying, and/or increased manifestation of panic and anxiety under treatment with benzodiazepines.

Here it is defined that a patient shows the clinical sign and symptom “family history of colorectal cancer” if patient or a first- or second-degree family member has ever received a medical diagnosis of “colorectal cancer” or shows symptoms fitting the diagnosis of “colorectal cancer”.

Here it is defined that a patient shows the clinical sign and symptom “family history of prostatic cancer” if patient or a first- or second-degree family member of the patient has ever received a medical diagnosis of “prostatic cancer” or shows symptoms fitting the diagnosis of “prostatic cancer”.

Herein, it is defined that a patient shows the clinical sign and symptom “hyperalgesia” if patient has increased sensitivity to pain or enhanced intensity of pain sensation.

In a preferred embodiment, an ASD phenotype 3 patients shows at least two, three or four of clinical signs and symptoms of the first group. In a particularly preferred embodiment, an ASD phenotype 3 patients shows “family history of hypertension” and at least one other clinical sign and symptom of the first group. This allows for a particularly reliable and easy identification of ASD phenotype 3 patients.

In another preferred embodiment, an ASD phenotype 3 patient does not show any of clinical signs and symptoms of the second group comprising hypokalemia, hypotension, hyperphosphatemia and hypochloraemia.

Herein, it is defined that a patient shows the clinical sign and symptom “hypokalemia” if the patient has a serum potassium level below 3.5 mEq/L (for children over the age of 2 years to adults; Simon et al. Hyperkalemia. St Treasure Island (FL): StatPearls Publishing).

Herein, it is defined that a patient shows the clinical sign and symptom “hypotension” if if the patient has as a systolic blood pressure (SBP) below 90 mm Hg, or a diastolic blood pressure (DBP) below 60 mm (for children over the age of 10 years to adult).

Herein, it is defined that a patient shows the clinical sign and symptom “hyperphosphatemia” if patient has a serum phosphate level above 4.5 mg/dL (adult) or above 7 mg/dL (children; Sharma et al. Hypophosphatemia. St Treasure Island (FL): StatPearls Publishing).

Herein, it is defined that a patient shows the clinical sign and symptom “hypochloraemia” if the patient has a serum chloride concentration below 90 mEq/L.

The combination of positive and negative clinical signs and symptoms allows for an even better differentiation between ASD phenotype 3 patients and other idiopathic ASD patients.

According to the invention, ASD phenotype 3 patients can be treated by and respond positively to administration of substances capable of stabilizing the resting membrane potential of excitable cells such as neuronal and pancreatic cells.

The person skilled in the art is aware how a substance is capable of inducing repolarization of the membrane of excitable cells after depolarization. For example, the substance may contribute to repolarization by balancing the ion concentration gradient by modulating voltage-gated cation channels activity through the transcriptional up-regulation of genes such as SNAP25, KCNJ6, KCNQ3, KCNK3, KCNJ10, and KCNV1. Alternatively, channel activity can also be modulated by down-regulation of GNAS. Likewise, substances decreasing the release of agonists, such as insulin, may also contribute to repolarization by the transcriptional up-regulation of SNAP25 and SLC12A5 (KCC2), and down-regulation of GNAS.

The person in the art therefore also knows how substances capable of contributing to repolarization the membrane of excitable cells after depolarization can be identified. For example, the person skilled in the art can identify such substances by testing whether the substance upregulates SNAP25, KCNJ6, KCNQ3, KCNK3, KCNJ10, and/or KCNV1 or downregulates GNAS and/or MIF. Such an analysis is within the routine measures of the skilled person.

Substances capable of inducing repolarization of the membrane of excitable cells after depolarization include loop diuretics and thiazide diuretics and may be selected from the groups of sulfonamides, in particular bumetanide, azosemide, benzmetanide, clofenamide, clopamide, clorexolone, furosemide, indapamide, mefruside, meticrane, metolazone, piretanide, torasemide (torsemide) and xipamide; meta-aminobenzoates; benzothiazides, in particular bendroflumethiazide, chlorothiazide, cyclopenthiazide, cyclothiazide, hydrochlorothiazide, hydroflumethiazide, mebutizide, methylclothiazide, polythiazide and trichlormethiazide; thiazoles, in particular etozolin, ozolinone and tizolemide; quinazolines, in particular fenquizone and quinethazone; phthalimides, in particular chlortalidone; phenoxyacetates, in particular etacrynic acid; indans, in particular indocrinone; pyrazoles, in particular muzomimine; indoles, in particular tripamide.

In a preferred embodiment, the substance capable of inducing repolarization of the membrane of excitable cells after depolarization is a sulfonamide.

In a preferred embodiment of the invention, the substance capable of inducing repolarization of the membrane of excitable cells after depolarization is bumetanide, AqB007, AqB011, PF-2178, BUM13, BUMS, bumepamine or a mixture thereof. In a particularly preferred embodiment, the substance capable of inducing repolarization of the membrane of excitable cells is bumetanide, also referred to as 3-(butylamino)-4-phenoxy-5-sulfamoylbenzoic acid, a potent loop diuretic.

The pharmaceutical composition according to the invention is to be administered in a therapeutically effective amount. For example, 0.01 to 10 mg of the pharmaceutical composition according to the invention may be administered in a single daily dose. In preferred embodiments, the pharmaceutical composition according to the invention is administered as a total daily dose of 0.5 to 10 mg, more preferably 0.5 to 6 mg, most preferably 0.5. to 4 mg either once or twice daily.

The person skilled in the art is aware of ways and methods to administer the pharmaceutical composition according to the invention. For example, the pharmaceutical composition according to the invention is administered orally, nasally, parenterally or intravenously. In a preferred embodiment, the pharmaceutical composition according to the invention is administered orally.

In order to achieve the desired dosage level, the pharmaceutical composition according to the invention may be administered once daily or in several doses per day.

In a preferred embodiment, the pharmaceutical composition according to the invention comprises bumetanide and is administered 0.5 to 2 mg orally once daily or 0.5. to 1 mg intravenously or intramuscularly. In one embodiment, the pharmaceutical composition is administered as a continuous intravenous infusion with 1 mg/hour up to 12 mg/day.

In another embodiment, the pharmaceutical composition is for use in the treatment of pediatric patients younger than 6 months, wherein the treatment comprises administration of 0.01 to 0.05 mg/kg, preferably 0.04 mg/kg bumetanide, once daily or every other day. In another embodiment the pharmaceutical composition is for use in the treatment of pediatric patients older than 6 months, wherein the treatment comprises administration of 0.015 to 0.1 mg/kg bumetanide once daily or every other day.

The pharmaceutical composition according to the invention may additionally comprise pharmacologically acceptable excipients such as fillers, disintegrants, lubricants, adhesives, anti-adherents, binders, coatings, glidants, sweeteners, flavors, sorbents, colors and preservatives. The person skilled in the art knows how to formulate an active ingredient in a way to facilitate drug absorption, reduce viscosity or enhance solubility.

In one embodiment, the pharmaceutical composition comprising a substance capable of stabilizing the resting membrane potential of neuronal and pancreatic cells is used in the treatment of ASD in a patient, wherein the treatment comprises
determining whether the patient suffers from ASD phenotype 3 and
administering a therapeutically effective amount of the pharmaceutical composition to the patient if the patient suffers from ASD phenotype 3,
   wherein determining whether the patient suffers from ASD phenotype 3 comprises checking for upregulation of GNAS and/or MIF, and/or checking for the downregulation of CTNNA2, GABRA1, GABBR2, NR3C1, SLC12A5, KCNJ6, KCNQ3, KCNJ10, DRD1 and/or SNAP25. Herein, GNAS refers to the heterotrimeric G-protein alpha subunit, MIF refers to macrophage migration inhibitory factor, CTNNA2 refers to alpha-2-catenin, GABRA1 refers to gamma-aminobutyric acid receptor subunit alpha-1, GABRA2 refers to gamma-aminobutyric acid receptor subunit alpha-2, NR3C1 refers to glucocorticoid receptor (nuclear receptor subfamily 3, group C, member 1), SLC12A5 refers to chloride potassium transporter member 5, KCNJ6 refers to G protein-activated inward rectifier potassium channel 2, KCNQ3 refers to Kv7.3 potassium channel, KCNJ10 refers to ATP-sensitive inward rectifier potassium channel 10, DRD1 refers to dopamine receptor D1, and SNAP25 refers to synaptosomal nerve-associated protein 25.

The skilled person is well aware how an analysis of upregulation and downregulation of specific genes can be performed. For example, it may be checked on the mRNA level using quantitative PCR techniques such as qPCR or RT-qPCR or semi-quantitative techniques such as RNA sequencing or Fluorescence In Situ Hybridization (FISH) techniques. Likewise, up- or downregulation of a gene may be determined on the protein level using protein quantification techniques such as Western Blot and quantitative dot blots. Upregulation is understood to mean an increase of mRNA levels or protein levels of at least 20% when compared to controls. Downregulation is understood to mean a decrease of mRNA levels or protein levels of at least 20% when compared to controls.

In another embodiment, it is determined that the patient suffers from ASD phenotype 3 if he shows at least two of the clinical signs and symptoms of the first group, namely gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia.

The present invention is also directed to a method for dividing an ASD patient cohort into patients responding to treatment with a substance capable of inducing repolarization of the membrane of excitable cells after depolarization and patients not responding to said treatment, comprising the steps of:
providing a patient database including data sets relating to the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia; and
classifying a patient as a patient responding to treatment with a substance capable of inducing repolarization of the membrane of excitable cells after depolarization, if he shows at least two of the clinical signs and symptoms of said first group.

Likewise, the invention is directed to a method for dividing an ASD patient cohort into patients responding to treatment with a substance capable of inducing repolarization of membrane of excitable cells after depolarization and patients not responding to said treatment, comprising the steps of:
checking for the expression of GNAS, MIF, CTNNA2, GABRA1, GABBR2, NR3C1, SLC12A5, KCNJ6, KCNQ3, KCNK3, KCNJ10, KCNV1, DRD1 and/or SNAP25 in biological samples of each patient in the patient cohort; and
classifying a patient as a patient responding to treatment with a substance capable of inducing repolarization of membrane of excitable cells after depolarization if he shows upregulation of GNAS and/or MIF, and/or downregulation of CTNNA2, GABRA1, GABBR2, NR3C1, SLC12A5, KCNJ6, KCNQ3, KCNK3, KCNJ10, KCNV1, DRD1 and/or SNAP25.

The biological sample may be any sample suitable for the determination of gene expression, in particular a liquid sample such as blood, plasma, saliva, urine or feces.

Herein, the term “respond” is understood to mean that a patient's condition improves upon administration of the treatment, whereas the term “non-responding” is meant to cover patients showing no improvement or even worsening of the condition upon administration. In particular, the term “respond” is defined as showing an improvement of ASD core and ancillary symptoms upon administration of the pharmaceutical composition according to the invention. ASD core symptoms include but are not limited to social-interaction difficulties, communication challenges and a tendency to engage in repetitive behavior. ASD ancillary symptoms include but are not limited to intellectual disability, learning disability, genitourinary organ dysfunction (i.e., impairment to initiate urinating), episodes of diarrhea.

The present invention is therefore useful for stratifying a general population of ASD patients into a subgroup that responds to treatment with a substance capable of inducing repolarization of the membrane of excitable cells after depolarization and a subgroup that does not respond to said treatment, in particular bumetanide. Such a stratification is advantageous when designing clinical studies because given the heterogeneous nature of ASD, many treatments will show efficacy only in a subgroup of patients. Therefore, if a non-stratified patient population is used for a clinical study, treatment efficacy in a subgroup of patients can be masked by non-efficacy in the rest of the patient population.

A response may also comprise a decrease in non-compliance/disruptive behaviors, repetitive behaviors and restricted interests (as measured by the ADI-R, ABC (Aberrant Behavior Checklist) specific subscales (i.e., ABC-I)) as well as worsening of preexisting qualitative and quantitative communication and social deficits as measured by Social Responsiveness Scale (SRS).

Particularly, responders to a treatment may display an increase in social responsiveness characterized by but not limited to decreased latency to respond or establish eye contact and/or improved initiation of speech measured by mean number of verbal initiation within a given time interval in a similar contextual environment, decreased latency to response to verbal initiation, increase in executive functioning including increased ability to plan and implement multiple step tasks, increase in behavioral compliance, decreased irritability (i.e., ABC irritability subscale), lowered sensitivity to sensory stimuli, increase in short term memory retention and marked decrease or improvement in intensity of idiosyncratic behaviors and postures.

A positive response may also comprise or consist of positive variation in scores in standardized tests such as:

Autism Diagnostic Interview-Revised (ADI-R): is a standardized, semi-structured clinician led parent interview. The ADI-R includes 93 items focusing on Early Development,

Language/Communication, Reciprocal Social Interactions, and Restricted, Repetitive Behaviors and Interests. (ADI-R; Rutter et al. 2003). It is divided into four different modules.

Aberrant Behavior Checklist (ABC): is a symptoms rating checklist used to assess and classify problem behaviors of children and adults in a variety of settings. The ABC includes 58 items that resolve into five subscales: (1) irritability, (2) lethargy/social withdrawal, (3) stereotypic behavior, (4) hyperactivity/noncompliance, and (5) inappropriate speech. A negative change signifies improvement and a positive change signifies worsening. The value for determining positive or negative response is the change from baseline to 3-5 days after administration of the pharmaceutical composition according to the present invention.

Social Responsiveness Scale (SRS). The 65-item SRS is a standardized measure of the core symptoms of autism. Each item is scored on a 4-point Likert scale. The score of each individual item is summed to create a total raw score. Total scores results are as follows:
0-62: Within normal limits
63-79: Mild range of impairment
80-108: Moderate range of impairment
109-149: Severe range of impairment

The value for determining positive or negative response is the change from baseline to 3-5 days after treatment according to the present invention. The total score ranges from 0-149. A negative change signifies improvement and a positive change signifies worsening.

Clinical Global Impression Severity Scale (CGI-S): The CGI-S is a 7-point scale that requires the clinician to rate the severity of the patient's illness at the time of assessment, relative to the clinician's past experience with patients who have the same diagnosis.

Considering total clinical experience, a patient is assessed on severity of mental illness at the time of rating:
1: normal, not at all ill
2: borderline mentally ill
3: mildly ill
4: moderately ill
5: markedly ill
6: severely ill
7: extremely ill

The value for determining positive or negative response is the change from baseline to 3-5 days after treatment according to the present invention. The total score ranges from 1-7. A negative change signifies improvement and a positive change signifies worsening.

Clinical Global Impression Improvement Scale (CGI-I): The CGI-I is a 7-point scale measure of overall change of Parent Target Problems (the child's two most pressing problems at screening, as reported by parents), using scores from the Clinical Global Impressions—Severity scale (CGI-S). Scores range from:
1: very much improved
2: much improved
3: minimally improved
4: no change
5: minimally worse
6: much worse
7: very much worse

The value for determining positive or negative response is the change from baseline to 3-5 days after treatment according to the present invention. The total score ranges from 1-7. A negative change signifies improvement and a positive change signifies worsening.

Autism Diagnostic Observation Schedule (ADOS): The ADOS is a semi-structured standardized assessment of communication and social interaction that is considered a gold-standard assessment of ASD. It is administered to diagnose autism and determine the level of severity of ASD in patients by evaluating impairments in two specific domains (the Social affect domain and the Restricted and Repetitive 25 behaviors domain). Different modules are used according to age and characteristics of the patients:
Module 1: children who use little or no phrase speech
Module 2: subjects who use phrase speech but do not speak fluently
Module 3: younger subjects who are verbally fluent
Module 4: adolescent and adults who are verbally fluent
   and involve expert clinical judgement of the qualities and behaviors that are at the core of the social/communicative deficits. A negative change signifies improvement and a positive change signifies worsening. The ADOS scoring algorithm consists in 2 raw domain scores that are calibrated on a single 10-point scale, the ADOS-CSS (Calibrated Severity Scores).

Individuals with scores between 6-10 on ADOS-CSS receive the classification “Autism”, those with a CSS of 4-5 “ASD” down to “Nonspectrum” for individuals with CSS of 1-3. The value for determining positive or negative response is the change from baseline to 3-5 days after administration of the Nrf2-activator pharmaceutical composition according to the present invention.

Childhood Autism rating Scales (CARS), (Schopler et al. Toward objective classification of childhood autism: Childhood Autism Rating Scale (CARS). J Autism Dev Disord. 1980 March; 10(1):91-103). The CARS consists of 14 domains assessing behaviors associated with autism, with a 15th domain rating general impressions of autism. Each domain is scored on a scale ranging from one to four; higher scores are associated with a higher level of impairment. Total scores can range from a low of 15 to a high of 60; scores below 30 indicate that the individual is in the non-autistic range, scores between 30 and 36.5 indicate mild to moderate autism, and scores from 37 to 60 indicate severe autism (Schopler et al. The Childhood autism rating sclares: (CARS). Loas Angeles, Calif.: Western Psychological Services, 1988).

Other means to measure a change in ASD symptoms include: mean length of utterance (MLU) defined as the number of words or morphemes in each of spontaneous utterances. This measure is one of the most robust indices of young children's language acquisition. MLU is used to diagnose language impairments in young children, often defined as an MLU level one standard deviation or more below the mean for the child's age level. Additionally, intellectual functioning as a core symptom of ASD may be measured via the Vineland Adaptive Behavior Scale II (VABS-II), wherein a raise in scores signifies improvement, i.e., positive response.

A positive response may also comprise a positive change in one or more indicators measuring core or ancillary symptoms of ASD including: changes in observer reported or computer monitored eye tracking revealing level of interest in social stimuli versus objects, engagement in and responsiveness to social interaction, verbal and nonverbal communication abilities, occurrence of repetitive behaviors, level of intellectual functioning, measurement of attention, quality of motor coordination, quality of sleep, and intensity of symptoms of medical comorbidities (e.g. gastro-intestinal symptoms).

For example, a positive response as defined herein may be identified by a decrease of at least 10%, 20%, 30%, 40%, 50%, 60%, 70%, 80%, or 90% in any of ADI-R ABC, ABC-I, SRS, CARS or ADOS scores. In a preferred embodiment, the positive response comprises a decrease of at least 10% in total scores in these scales or any increase corresponding to a “minimally improved” (3) and preferably “much improved” (2) or “very much improved” (1) rating on the CGI-I scale.

In another embodiment, the present invention is directed to a method for diagnosing a patient with ASD phenotype 3, comprising the steps of:
providing a patient-specific data set relating to the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia; and
diagnosing the patient with ASD phenotype 3 if he shows at least two of the clinical signs and symptoms of said first group.

In yet another embodiment, the present invention is directed to a method for diagnosing an ASD patient with ASD phenotype 3, comprising the steps of:
checking for the expression of GNAS, MIF, CTNNA2, GABRA1, GABBR2, NR3C1, SLC12A5, KCNJ6, KCNQ3, KCNK3, KCNJ10, KCNV1, DRD1 and/or SNAP25 in biological sample of the patient; and
diagnosing the patient as ASD phenotype 3 if he shows upregulation of GNAS and/or MIF, and/or downregulation of CTNNA2, GABRA1, GABBR2, NR3C1, SLC12A5, KCNJ6, KCNQ3, KCNK3, KCNJ10, KCNV1, DRD1 and/or SNAP25.

In a preferred embodiment, a patient is diagnosed or classified as ASD phenotype 3 if he shows two of the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia

In a preferred embodiment, a patient is diagnosed or classified as ASD phenotype 3 if he shows “family history of hypertension” and at least one other of the clinical signs and symptoms of said first group. In another embodiment, a patient is diagnosed or classified as ASD phenotype 3 if he shows at least three of the clinical signs and symptoms of said first group.

### EXAMPLES

### Example 1—Identification of Differentially Expressed Genes

Material and Methods

Profiling drugs across a variety of these cell lines and treatment conditions (such as dose and time) provides a representative biological context for characterizing the cell line invariant mechanism of action as demonstrated previously (Iorio et. al. Discovery of drug mode of action and drug repositioning from transcriptional responses, Proc Natl Acad Sci USA. 107(33):14621-6, 2010 Aug. 17; lorio et. al. Transcriptional data: a new gateway to drug repositioning? Drug Discovery Today, 18, 7-8, 2013; Carrella D et al. Mantra 2.0: an online collaborative resource for drug mode of action and repurposing by network analysis, Bioinformatics 30 (12): 1787-1788, 2014; Sirci et. al. Computational Drug Networks: a computational approach to elucidate drug mode of action and to facilitate drug repositioning for neurodegenerative diseases, Drug Discovery Today: Disease Model, 19, 11-17, 2016; Pushpakom et. al. Drug repurposing: progress, challenges and recommendations, Nature reviews Drug discovery 18, 1, 41-58, 2019).

To characterize the cell line invariant mechanism of action, bumetanide was tested across different human cell lines including several cancer cell lines (such as A549, MCF7, PC3, HT29 and VAP) as well as cells from healthy tissues (such as ASC and HA1E). These cell lines (Table 3) were chosen due to their ease of manipulation and suitability for systematic screening. The growth medium used for the cell lines varied according to the different cell type characteristics, as suggested by the cell purchaser company recommendations. Incubation of cell types was performed inside an incubator with a humified atmosphere of 95% and 5% of CO2, and a constant temperature of 37° C.

Cells were treated with bumetanide using a dilution of 10 uM for all replicates at a time duration of 6 or 24 hours.

TABLE 3
Time duration and dosage used for treating cell lines with bumetanide

| Cell line | Cell type | Reference | Time duration | Dosage |
|---|---|---|---|---|
| A569 | Non-small cell | ATCC CCL-185 | 6 hours | 10 uM |
| | lung carcinoma | | | |
| ASC | Adipocyte | From Jeff Gimble | 24 hours | 10 uM |
| | | MD PhD, PBRC | | |
| HA1E | Kidney | CVCL_VU89 | 6 hours | 10 uM |
| HT29 | Intestine | ATCC HTB-38 | 6 hours | 10 uM |
| MCF7 | Breast | ATCC HTB-22 | 6 hours | 10 uM |
| | adenocarcioma | | | |
| PC3 | Prostate | ATCC CRL-1435 | 24 hours | 10 uM |
| | adenocarcinoma | | | |
| VCAP | Prostate | ATCC CRL-2876 | 6 hours | 10 uM |
| | carcinoma | | | |

Following drug treatment, RNA extracted from each treated cell line was sequenced with QuantSeq with 4-6 million coverage reads per sample in triplicates. Next, differential gene gxpression analysis (DGE) was performed in order to evaluate which genes showed the highest expression variability following bumetanide treatment.

Finally, STALICLA's proprietary HC-Match module (EP 20164353) was applied to conduct gene set enrichment analyses comparing genes found to be dysregulated by bumetanide and genes whose deficiency or excess is associated with specific clinical signs and symptoms included in STALICLA's proprietary and ASD-tailored knowledge bases. This analysis allowed us to identify a subset of ASD-related clinical signs and symptoms describing patients that can benefit from bumetanide treatment, given a corrective effect at the gene expression level for such endophenotypes.

Results

The ranking of genes for which up- or downregulation of expression is associated with the capacity to repolarize the membrane of excitable cells after depolarization is listed in Table 4.

TABLE 4
Ranking of the differential expressed genes (DEG)
after bumetanide treatment in cell lines.

| | DEG ranking | Direction of |
|---|---|---|
| Gene | after treatment | differential expression |
| SNAP25 | 1 | UP |
| KCNJ6 | 19 | UP |
| SLC12A5 | 35 | UP |
| (KCC2) | | |
| KCNQ3 | 48 | UP |
| KCNK3 | 49 | UP |
| KCNJ10 | 113 | UP |
| KCNV1 | 165 | UP |
| GNAS | 1 | DOWN |

These results provide evidence for bumetanide having the capacity to contribute to repolarize excitable cells after depolarization mainly by:
1. upregulating genes that encode for potassium voltage-gated channels whose main function is to contribute to repolarize excitable cells after depolarization, including KCNQ3, KCNK3, and KCNV1, as well as inward-rectifier potassium channels that help to restore the cells resting membrane potential, including KCNJ6 and KCNJ10;
2. regulating genes that have the capacity to decrease calcium influx and therefore shorten the duration of action potentials and decrease the release of neurotransmitters and hormones that can further trigger depolarization of excitable cells (Mansvelder et al. The relation of exocytosis and rapid endocytosis to calcium entry evoked by short repetitive depolarizing pulses in rat melanotropic cells. J Neurosci. 1998 Jan. 1; 18(1):81-92; Mansvelder et al. All classes of calcium channel couple with equal efficiency to exocytosis in rat melanotropes, inducing linear stimulus-secretion coupling. J Physiol. 2000 Jul. 15; 526 Pt 2:327-39), through the transcriptional up-regulation of genes such as SNAP25 and SLC12A5, and the transcriptional down-regulation of GNAS.

### Example 2—Observational Clinical Trial Pilot Study

This pilot study aimed at investigating the clinical benefit of a pharmaceutical composition comprising a substance capable of inducing repolarization of the membrane of excitable cells after depolarization for use in the treatment of autism spectrum disorder (ASD) phenotype 3 (ASD-Phen3).

Material and Methods

Three patients with idiopathic ASD that had previously shown improvement after receiving an oral solution of bumetanide at a dosage of 0.5, 1.0, or 2 mg twice daily for three months were enrolled in the study. The main goal of this pilot study was to validate the clinical benefit of bumetanide in patients showing clinical signs and symptoms characterizing ASD phenotype 3.

First, a custom semi-structured online clinical questionnaire (Questionnaire Q3© STALICLA SA 2021) comprising a total of 38 medical examination questions and 73 medical history questions was administered by a clinician during a remote screening visit (over the phone) that took up to 90 min. This remote screening visit included reviewing detailed medical history and clinical signs and symptoms related to ASD phenotype 3 with the patient/caregiver for a clinical validation. In particular, the clinical questionnaire contains questions regarding
the presence of clinical signs and symptoms from the first group comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia, and
the absence of clinical signs and symptoms from the second group comprising hypokalemia, hypotension, hyperphosphatemia and hypochloraemia.

Results

As illustrated in Table 5, all three patients showed at least 2 of the first group of clinical signs and symptoms and none of the second group of clinical signs and symptoms (CSS). In addition, two out of the three enrolled bumetanide responder patients had a family history of hypertension and at least one other of the first group of clinical signs and symptoms.

TABLE 5
Summary results from the observational clinical trial pilot study

| | Number of CSS of | Family history of | Number of CSS of |
|---|---|---|---|
| | the first group | hypertension | the second group |
| Patient #1 | 7/25 | No | 0/4 |
| Patient #2 | 11/25 | Yes | 0/4 |
| Patient #3 | 10/25 | Yes | 0/4 |

### Example 3—Observational Clinical Trial

This study aims at investigating the clinical benefit of a pharmaceutical composition comprising a substance capable of inducing repolarization of the membrane of excitable cells after depolarization for use in the treatment of autism spectrum disorder (ASD) phenotype 3 (ASD-Phen3).

A maximum of 88 patients with idiopathic ASD that had previously received an oral solution of bumetanide at a dosage of 0.5, 1.0, or 2 mg twice daily for three months are enrolled in the study. This includes patients that showed improvement under this treatment, as well as those that showed no response. The main goal of this observational study is to provide evidence of the existence of a well characterized subgroup of patients with ASD also referred to as ASD phenotype 3, as a target population showing positive responses to treatment with bumetanide.

First, a custom semi-structured online clinical questionnaire (Questionnaire Q3© STALICLA SA 2021) comprising a total of 38 medical examination questions and 73 medical history questions is administered by a clinician during a remote screening visit (over the phone) that takes up to 90 min. This remote screening visit includes reviewing detailed medical history and clinical signs and symptoms related to ASD phenotype 3 with the patient/caregiver for a clinical validation. In particular, the clinical questionnaire contains questions regarding
the presence of clinical signs and symptoms from the first group comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia, and
the absence of clinical signs and symptoms from the second group comprising hypokalemia, hypotension, hyperphosphatemia and hypochloraemia.

The remote screening is followed by an in-person visit for blood draw and completion of the clinical questionnaire. Blood draws are performed to collect multi-omics data (including metabolomics, transcriptomics (RNA-seq) and whole-genome sequencing data) and to obtain a biological validation of ASD phenotype 3.

In order to validate the existence of ASD phenotype 3, the number of enrolled patients showing at least two of the first group of clinical signs and symptoms (group 1) as well as the proportion of responders (i.e., patients having shown an improvement of ASD core and ancillary symptoms following treatment with bumetanide) and non-responders (i.e., patients having shown a worsening of ASD core and ancillary symptoms under treatment with bumetanide) within this patient group is assessed.

Furthermore, the number of enrolled patients showing hypertension and at least one other of the first group of clinical signs and symptoms (group 2) as well as the proportion of responders and non-responders within this patient group is assessed.

In addition, in each group, the status regarding the second group of clinical signs and symptoms is assessed.

The proportion of responders in each of the groups is then compared to the proportion of responders within all patients enrolled in the study. It is expected that the proportion of responders is much higher in groups 1 and 2 than in the non-stratified group comprising all enrolled patients.

Furthermore, it is expected that the status regarding the second group of signs and symptoms allows to further differentiate between responders and non-responders.

In order to further validate the proposed disease mechanism, samples from patients are assessed for upregulation of GNAS and/or MIF, and/or downregulation of CTNNA2, GABRA1, GABBR2, NR3C1, SLC12A5, KCNJ6, KCNQ3, KCNK3, KCNJ10, KCNV1, DRD1 and/or SNAP25. Patients are then divided into different groups based on their status for the above mentioned genes and the proportions of responders and non-responders to treatment with bumetanide in each group are calculated.

1. A method
(a) for the treatment of ASD in a patient, wherein the treatment comprises
administering a therapeutically effective amount of a pharmaceutical composition comprising a substance capable of inducing repolarization of membrane of excitable cells after depolarization to the patient wherein the patient has upregulation of GNAS and/or MIF, and/or checking for the downregulation of CTNNA2, GABRA1, GABBR2, NR3C1, SLC12A5, KCNJ6, KCNQ3, KCNK3, KCNJ10, KCNV1, DRD1 and/or SNAP25,
or
(b) for the treatment of ASD in a patient, wherein the treatment comprises
administering a therapeutically effective amount of a pharmaceutical composition comprising a substance capable of inducing repolarization of membrane of excitable cells after depolarization to the patient that shows at least two of the following first group of clinical signs and symptoms comprising gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia.

2. (canceled)

3. The method according to claim 1, wherein the patient does not show any of the following second group of clinical signs and symptoms comprising hypokalemia, hypotension, hyperphosphatemia and hypochloraemia.

4. (canceled)

5. (canceled)

6. The method according to claim 1, therein method (a) or (b), wherein the excitable cells are neuronal cells and beta pancreatic cells.

7. The method according to claim 1, therein method (a) or (b), wherein the substance capable of inducing repolarization of the membrane of excitable cells after depolarization is selected from the group of sulfanilamides, in particular azosemide, benzmetanide, clofenamide, clopamide, clorexolone, furosemide, indapamide, mefruside, metolazone, piretanide, torasemide (torsemide) and xipamide; meta-aminobenzoates, in particular bumetanide; benzothiadiazines, in particular bendroflumethiazide, chlorothiazide, cyclopenthiazide, cyclothiazide, hydrochlorothiazide, hydroflumethiazide, mebutizide, methylclothiazide, polythiazide and trichlormethiazide; thiazoles, in particular etozolin, ozolinone and tizolemide; quinazolines, in particular fenquizone and quinethazone; phthalimides, in particular chlortalidone; phenoxyacetates, in particular etacrynic acid; indans, in particular indocrinone; pyrazoles, in particular muzomimine; and indoles, in particular tripamide.

8. The method according to claim 1, therein method (a) or (b), wherein the substance capable of inducing repolarization of the membrane of excitable cells after depolarization is a derivative of 4-substituted-3-amino-5-sulfamoylbenzoic acid.

9. The method according to claim 8, wherein the derivative of 4-substituted-3-amino-5-sulfamoylbenzoic acid is selected from the group consisting of bumetanide, AqB007, AqB011, PF-2178, BUM13, BUMS, bumepamine and mixtures thereof.

10. The method according to claim 1, therein method (a) or (b), wherein the patient shows a family history of hypertension and at least one other of the clinical signs and symptoms.

11. The method according to claim 1, therein method (a) or (b), wherein the patient shows at least three of the first group of clinical signs and symptoms.

12. The method according to claim 1, therein method (a) or (b), wherein the substance capable of inducing repolarization of the membrane of excitable cells after depolarization is administered orally to the patient at a daily total dosage of between 0.01 to 10 mg total per day, preferably between 0.5 and 4 mg total a day.

13. A sample comprising
bodily fluid from a patient and a primer to GNAS, MIF, CTNNA2, GABRA1, GABBR2, NR3C1, SLC12A5, KCNJ6, KCNQ3, KCNK3, KCNJ10, KCNV1, DRD1 and/or SNAP25 wherein said patient has been diagnosed with ASD and has been treated with a substance capable of inducing repolarization of membrane of excitable cells after depolarization, and wherein said patient bodily fluid includes upregulated GNAS and/or MIF, and/or downregulated CTNNA2, GABRA1, GABBR2, NR3C1, SLC12A5, KCNJ6, KCNQ3, KCNK3, KCNJ10, KCNV1, DRD1 and/or SNAP25 as compared to controls.

14. A Method for preparing a sample, comprising the steps of:
receiving bodily fluid from a patient who has been diagnosed with ASD and combining it with a primer to GNAS, MIF, CTNNA2, GABRA1, GABBR2, NR3C1, SLC12A5, KCNJ6, KCNQ3, KCNK3, KCNJ10, KCNV1, DRD1 and/or SNAP25, wherein GNAS and/or MIF in said sample are upregulated as compared to controls, and/or downregulation of CTNNA2, GABRA1, GABBR2, NR3C1, SLC12A5, KCNJ6, KCNQ3, KCNK3, KCNJ10, KCNV1, DRD1 and/or SNAP25 are downregulated as compared to controls.

15. The method according to claim 13, wherein the ASD patient additionally shows at least two of the following first group of clinical signs and symptoms, said first group of clinical signs and symptoms consisting of gastric ulcer, hyperkinesia, hyperventilation, tachycardia, short neck, hypophosphatemia, McCune-Albright syndrome, seizures, family history of hypertension, hyperkalemia, gastroesophageal reflux, precocious puberty, hyperinsulinemia, increased inflammatory response, family history of cholangiocarcinoma, hearing impairment, hyperchloraemia, family history of diabetes, joint hypermobility, constipation, family history of breast cancer, worsening of ASD core symptoms under benzodiazepines, family history of colorectal cancer, family history of prostatic cancer and hyperalgesia.

16. The method according to claim 13, wherein the ASD patient additionally shows at least a family history of hypertension and one other of the clinical signs and symptoms of said first group.
